(19) United States
(12) Patent Application Publication
(10) Pub. No.: US 2003/0120151 A1
(43) Pub. Date: Jun. 26, 2003

(54) Magnetic resonance imaging methods and compositions

(75) Inventor: Chris D. Constantinides, Rockville, MD (US)

Correspondence Address: EDWARDS & ANGELL, LLP, P.O. BOX 9169, BOSTON, MA, 02209, US

(73) Assignee: Johns Hopkins University, Baltimore, MD (US)

(21) Appl. No.: 10/044,296

(22) Filed: Jan. 10, 2002

Related U.S. Application Data
(60) Provisional application No. 60/260,524, filed on Jan. 10, 2001.

(51) Int. Cl. A61B 5/055

(52) U.S. Cl. 600/431

(57) ABSTRACT

Methods and compositions are provided for magnetic resonance imaging of biological tissue, particularly methods and compositions for ²³Na and ³⁹K magnetic resonance imaging of cardiac tissue. In particular, methods of ²³Na magnetic resonance imaging (MRI) cardiac imaging is presented for attenuating ²³Na signals corresponding to ventricular cavity blood and viable well-perfused tissue and for visualizing myocardial infarction. Cardiac tissue is imaged using ²³Na MRI after the introduction of an intravascular paramagnetic contrast agent. Optimally, the intravascular paramagnetic contrast agent is MION-46. The MION-46 suppresses the blood signal intensity in sodium images of ventricular cavities and signal for viable cardiac tissue. The quantity of MION-46 and the echo time (TE) for ²³Na MRI of cardiac tissue may be selected to minimize signal intensity differences between ventricular cavity blood and well-perfused viable myocardium; maximize signal intensity differences between non-viable myocardium and ventricular cavity blood in myocardial infarction; and maximize signal intensity differences between non-viable myocardium and well-perfused viable myocardium in myocardial infarction.

The present application claims the benefit of U.S. provisional application No. 60/260,524 filed Jan. 10, 2001, which is incorporated by referenced in its entirety.

Funding for the present invention was provided in part by the Government of the United States by virtue of a grant (HL 61695) by the National Institutes of Health. Accordingly, the Government of the United States has certain rights in this invention.

### BACKGROUND

1. Field of the Invention.

The present invention relates to methods of magnetic resonance imaging (MRI), including use of new contrast agents. Methods and compositions of the invention are especially useful for imaging of cardiac tissue and, in particular, to the use of a paramagnetic contrast agent to decrease contrast and thereby allowing delineation between blood cavities (i.e., atrial and ventricular) and viable cardiac tissue, and non-viable, infarcted cardiac tissue.

2. Background of the Invention.

Certain intravascular contrast agents have been used in proton (¹H) magnetic resonance imaging (MRI) of the cardiovascular system. In addition, certain iron oxide particles have been reported for use as MRI contrast agents for imaging the liver and spleen.

Sodium (²³Na) magnetic resonance imaging (MRI) has been used to detect signal elevations associated with nonviable myocardium after acute infarction and reperfusion. ²³Na MRI is uniquely capable of detecting altered sodium levels associated with impaired sodium-potassium pump function and nonviable tissue after acute infarction and reperfusion. Clinical applications of ²³Na MRI, however, are limited by the low in vivo concentrations and NMR sensitivity of endogenous sodium, which translates to coarse spatial resolution and lower sensitivity compared to the ¹H MRI. ³⁹K MRI imaging suffers some similar problems. See, generally, U.S. Pat. Nos. 5,910,112 and 6,205,349.

In the heart, the problem of low spatial resolution of ²³Na MRI images is further compromised by the high sodium content of ventricular blood which hinders differentiation of the ventricular wall from the ventricular cavity, and hence the differentiation of elevated sodium in myocardial infarction (MI). Thus, there tends to be minimal contrast between the ventricular cavity and infarcted cardiac tissue.

Several alternate ¹H MRI solutions could be adopted to overcome the inability to differentiate between the ventricular wall and the ventricular cavity due to the high sodium content of ventricular blood in ²³Na MRI. Such methods include black-blood ¹H MRI gradient echo and various spin dephasing imaging techniques. While these methods can attenuate ventricular sodium blood signals, these methods are associated with increased echo times (TE) or the need for cardiac gating, leading to detrimental signal-to-noise ratio (SNR) losses and significant prolongation of the total image acquisition times. Longer TE times lead to increased susceptibility induced artifacts and decreased SNR values in ²³Na images.

An additional disadvantage with these existing ¹H MRI imaging methods is that the close proximity of the longitudinal (T₁), fast (T₂,) and slow (T_{2f}) sodium transverse relaxation times of blood and myocardium preclude the use of conventional inversion recovery or spin-echo techniques to attenuate or null ventricular blood signals.

It thus would be desirable to have new methods for assessing biological tissue. It would be particularly desirable to have new methods for assessing cardiac tissue, especially through ²³Na or ³⁹K imaging techniques.

### SUMMARY OF THE INVENTION

We have now discovered new methods and compositions for magnetic resonance imaging of biological tissue, particularly methods and compositions for ²³Na and ³⁹K magnetic resonance imaging of cardiac tissue.

More particularly, methods of the invention include use of an iron oxide contrast imaging agent in a variety of magnetic resonance imaging applications, particularly ²³Na and ³⁹K imaging of cardiac and other biological tissue. Preferred contrast agents for use in accordance with the invention comprise an iron atom coordinated with an organic polymer that has oxygen substitution, e.g. a polysaccharide such as a dextran.

Particularly preferred methods of the invention using an intravascular paramagnetic contrast agent to reduce or null sodium blood signals and to decrease the contrast between blood in the ventricular cavities and myocardial infarction using ²³Na MRI.

Preferred contrast agents of the invention comprise one or more iron atoms present in a complex, particularly complexed with one or more polymeric compounds. Oxygen-containing polymers are typically preferred, such as polysaccharides, particularly a dextran.

A particularly preferred intravascular paramagnetic contrast agent for use in accordance with the invention may comprise superparamagnetic intravascular iron oxide, T₂-relaxant contrast agent, MION-46. Optionally, the quantity of MION-46 introduced and the echo time (TE) are selected to minimize signal intensity differences between ventricular cavity blood and well-perfused viable myocardium; maximize signal intensity differences between non-viable myocardium and ventricular cavity blood in myocardial infarction; and maximize signal intensity differences between non-viable myocardium and well-perfused viable myocardium in myocardial infarction.

The invention, in one form thereof, is a method of ²³Na magnetic resonance imaging (MRI) of cardiac tissue for attenuating ventricular cavity blood signals and signals from viable perfused myocardial tissue and for visualizing non-invasively myocardial infarction. The method comprises introducing an intravascular paramagnetic contrast agent to attenuate the ²³Na MRI signal for ventricular cavity blood and viable well-perfused tissue. The cardiac tissue is then imaged using ²³Na MRI.

In yet a further embodiment, the method further comprises selecting a quantity of the MION-46 to be introduced and an echo time or imaging the cardiac tissue to minimize signal intensity differences between ventricular cavity blood and viable well-perfused myocardial tissue; maximize signal intensity differences between nonviable myocardium and ventricular cavity blood in myocardial infarction; and maximize signal intensity differences between non-viable myocardium and well-perfused viable myocardium in myocardial infarction.

The invention, in another form thereof, is a method for detecting myocardial infarction in cardiac tissue using ²³Na MRI comprising selecting a quantity of an intravascular paramagnetic contrast agent to be introduced and an echo time for imaging the cardiac tissue to minimize signal intensity differences between ventricular cavity blood and well-perfused viable myocardium; maximize signal intensity differences between non-viable myocardium and ventricular cavity blood in myocardial infarction; and maximize signal intensity differences between non-viable myocardium and well-perfused viable myocardium in myocardial infarction. The intravascular paramagnetic contrast agent is introduced to thereby attenuate ventricular cavity blood signals and viable well-perfused tissue. Next, the cardiac tissue is imaged using ²³Na MRI.

The invention, in yet another form thereof, is a method for detecting myocardial infarction in cardiac tissue comprising selecting a quantity of an intervascular paramagnetic contrast agent to be introduced and an echo time for imaging the cardiac tissue to minimize signal intensity differences between the ventricular cavity blood and viable well-perfused myocardium. The intravascular paramagnetic contrast agent is introduced to thereby attenuate ventricular cavity blood signals and ²³Na MRI signals from viable well-perfused tissue. Next, the cardiac tissue is imaged using ²³Na MRI.

The invention also includes evaluation of MION-46, an intravascular superparamagnetic contrast agent as a sodium MRI transverse (T₂) relaxant in an effort to attenuate the intense sodium blood signal from the ventricular cavity and for enhancing the ²³Na MRI contrast in MI.

The invention also provides methods and compositions to visualize cardiac tissue to detect myocardial infarction by using an intravascular paramagnetic contrast agent to attenuate a sodium blood signal in ²³Na MRI.

The invention further provides methods and compositions to decrease the contrast of the ventricular muscle wall and ventricular cavity blood in myocardial infarction using ²³Na MRI.

The invention also provides methods and compositions to select an appropriate dose of the paramagnetic contrast agent to decrease the contrast between the ventricular cavity blood and viable well-perfused tissue, thereby delineating myocardial infarction, using ²³Na magnetic resonance imaging (MRI).

The invention still further provides methods and compositions to select an echo time (TE) in ²³Na magnetic resonance imaging which uses a paramagnetic contrast agent, to minimize signal intensity differences between ventricular cavity blood and well-perfused viable myocardium; maximize signal intensity differences between non-viable myocardium and ventricular cavity blood in myocardial infarction; and maximize signal intensity differences between non-viable myocardium and well-perfused viable myocardium in myocardial infarction.

The invention yet further provides methods and compositions to select a dose of paramagnetic intravascular contrast agent and an echo time to decrease the signal intensity differences between ventricular cavity blood and viable well-perfused cardiac tissue while maximizing the signal intensity differences between non-viable myocardium and ventricular cavity blood in myocardial infarction and maximizing signal intensity differences between non-viable myocardium and well-perfused viable myocardium and non-viable myocardium in myocardial infarction.

Subjects that may be identified or diagnosed in accordance with the invention include mammals, particularly primates especially humans, that may be suffering from, suffered from or suspected of suffering from a disorder or injury associated with the heart, e.g. cardiac injury such as may be associated with heart failure, cardiogenic shock, cardiac ischemia, or a cardiovascular disease such any of a myocardial ischemic disorder, e.g. coronary artery disease, including angina pectoris and myocardial infarction.

Identification or diagnosis methods of the invention may suitably comprise administering an imaging effective amount of a contrast agent in accordance with the present invention and imaging targeted tissue of the subject with ²³Na or ³⁹K magnetic resonance, particularly by imaging cardiac tissue.

Other aspects of the invention are disclosed infra.

### BRIEF DESCRIPTION OF THE DRAWING

FIG. 1 illustratively depicts ²³Na MRI of a cross-section of an infracted heart without the administration of a contrast agent;

FIG. 2 illustratively depicts ²³Na MRI of a cross-section of an infarcted heart after the administration of a contrast agent according to the present invention;

FIG. 3 illustratively depicts ²³Na MRI of a cross-section of an infracted heart after administration of a contrast agent at the target dose and optimum echo time (TE);

FIG. 4 is a flow diagram schematically depicting a method of ²³Na MRI of cardiac tissue using a contrast agent according to the present invention;

FIG. 5 is a plot depicting normalized ²³Na MRI signal intensity variation with MION-46 volume at various echo times (TE);

FIG. 6A is a plot of T₁ and FIG. 6B is a plot T₂ vs. MION-46 volume;

FIGS. 7A-7E illustratively depict coronal of images of a gel-blood phantom;

FIG. 8A illustratively depicts pre-contrast axial images from a normal heart at optimal echo time of 5 ms and FIG. 8B illustratively depicts post-contrast axial images from a normal heart at target dose/optimal echo time of 5 ms;

FIG. 9 is a plot depicting ²³Na MRI signal intensity variation of myocardial tissue and ventricular blood regions with different MION-46 volume doses in vivo; and

FIG. 10A illustratively depicts pre-contrast ²³Na MRI images of an infarcted heart, FIGS. 10B-10C illustratively depict post contrast ²³Na MRI images of an infarcted heart and FIG. 10D illustratively depicts a TTC stained slice of the excised heart.

### DETAILED DESCRIPTION OF THE INVENTION

As discussed above, we have now found new methods and compositions for magnetic resonance imaging of biological tissue, particularly methods and compositions for ²³Na and ³⁹K magnetic resonance imaging of cardiac tissue.

The exemplary disclosure which follows often is made with respect to preferred contrast agent of MION-46 and ²³Na imaging. However, it is understood that disclosure is exemplary only, and other embodiments are fully within the scope of the invention as discussed herein, e.g. other contrast agents, magnetic resonance imaging other than ²³Na, and the like.

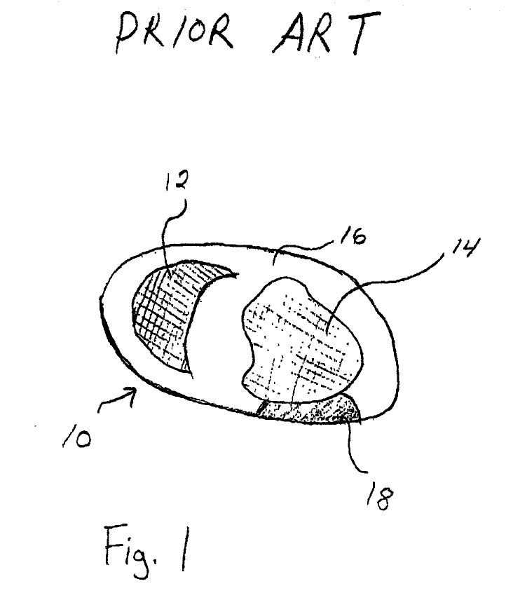

Referring now to the figures, FIG. 1 depicts a ²³Na MRI of a cross-section of infracted heart 10. Infarcted heart 10 is used for exemplary purposes to illustrate convention of ²³Na MRI of a cardiac tissue. Infarcted heart 10 Includes right ventricular cavity 12, left ventricular cavity 14 and ventricular wall 16. Infarcted heart 10 includes nonviable, infarcted myocardium 18 associated with a myocardial infarction.

²³Na MRI signal elevations are associated with nonviable myocardium 18. The elevated signal is the result of acute infarction followed by reperfusion of blood. Stated more precisely, ²³Na MRI detects myocardial infarction through altered sodium levels associated with cardiac tissue impaired sodium-potassium pump function of non-viable tissue after acute infarction and reperfusion.

In addition to the infarcted myocardial tissue 18, ²³Na MRI produces an intense signal for ventricular blood present in ventricular cavities 12, 14. As a result, there is minimal signal intensity differences between the ²³Na MRI signal for the infarcted myocardial tissue 18 and the ventricular cavities 12, 14. The lack of signal intensity differences between infarcted myocardial tissue 18 and the ventricular cavities 12, 14 may hamper ²³Na MRI detection of infarcted myocardium tissue 18.

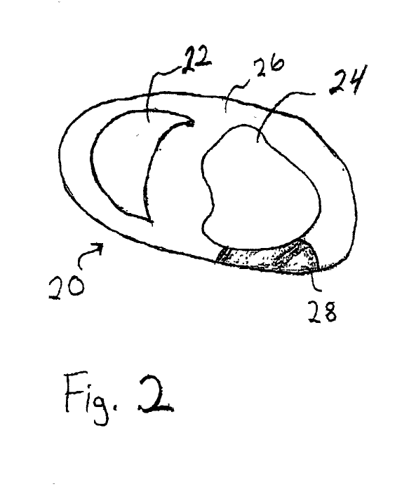

FIG. 2 depicts infarcted heart 20 after the administration of an intravascular paramagnetic contrast agent. Optimally, the intravascular paramagnetic contrast agent comprises superparamagnetic iron oxide such as MION-46. One manufacturer of MION-46 is Advanced Magnetics Inc. of Cambridge, Mass.

MION-46 has an average core size of 4-7 nm, and contains 27% iron by weight. MION-46 contains one central iron oxide crystal to which multiple dextran molecules are attached, increasing the diameter of the agent to about 17 nm. The MION-46 particles are scavenged from the blood by the reticuloendothelial system, especially the kupffer cells of the liver and have a half-life in the blood of about 81 minutes.

The MION-46 produces a hypointense signal in the ventricular cavities 22, 24 due to the paramagnetic effect of MION-46. As such, there is a reduction in intensity of ²³Na MRI signal from ventricular cavities 22, 24 in the presence of MION-46 as compared with signal associated with ventricular cavities 12, 14 in the absence of MION-46 (see FIG. 1).

The MION-46 may also produce a hypointense or slight reduction in ²³Na MRI signal corresponding to viable tissue in the ventricular wall 26. At high doses of MION-46, capillary blood may supply MION-46 at sufficient quantity to produce a reduction in ²³Na MRI signal for ventricular wall 26. The reduction in ²³Na MRI signal in the ventricular cavities 22, 24 results in enhanced signal intensity differences of non-viable infracted myocardial tissue 28. As a result, infarcted myocardial tissue 28 becomes more readily detected.

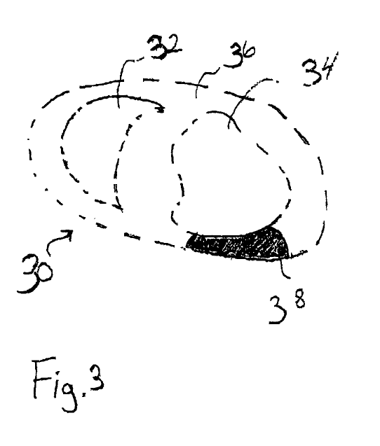

Referring now to FIG. 3, when the MION-46 dose (i.e. target dose) and TE are optimized for minimize signal intensity differences between ventricular cavity blood and well-perfused viable myocardium; maximize signal intensity differences between non-viable myocardium and ventricular cavity blood in myocardial infarction; and maximize signal intensity differences between non-viable myocardium and well-perfused viable myocardium in myocardial infarction, there is a hypointense signal in both ventricular cavities 33, 34 and ventricular wall 36 and a hypointense signal for the non-viable infarcted myocardium 38. Consequently, the major ²³Na MRI signals correspond to the non-viable infarcted myocardium 38.

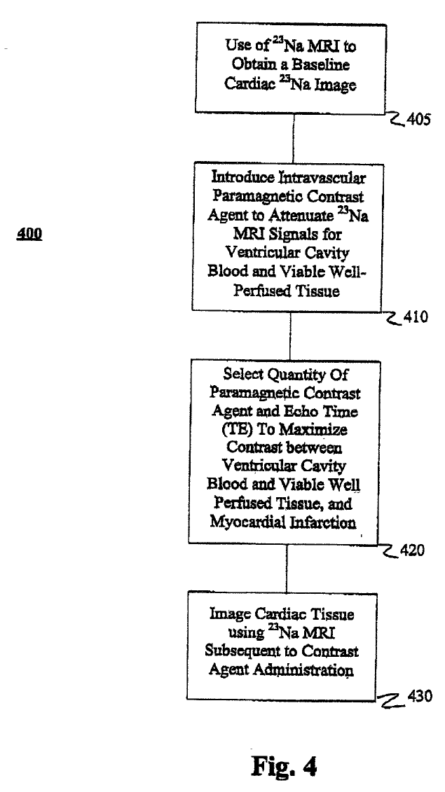

Referring now to FIG. 4, method 400 comprises the use of an intravascular paramagnetic contrast agent to attenuate sodium blood signals and to enhance the signal intensity differences between infarcted cardiac tissue and ventricular cavity blood in myocardial infarction using ²³Na MRI. A baseline ²³Na MRI is performed on the heart of a subject (step 405), particularly a mammal, such as a human patient.

An intravascular paramagnetic contrast agent is introduced intravenously to attenuate ²³Na MRI signals for ventricular cavity blood and viable well-perfused tissue (step 410). Optimally, the intravascular paramagnetic contrast agent comprises superparamagnetic iron oxide MION-46.

In order to maximize the contrast between the ventricular cavities 32, 34 and ventricular wall 36, and the non-viable infarcted myocardial tissue 38, an appropriate (i.e. target) dose of MION-46 is selected and the echo time (TE) is selectively chosen (step 420). It was discovered that minimum signal intensity differences between blood in the ventricular cavities 32, 34 and ventricular tissue 36, and maximum signal intensity differences between non-viable myocardium and ventricular cavity blood in myocardial infarction (MI); and maximum signal intensity differences between non-viable myocardium and well-perfused viable myocardium in myocardial infarction myocardial tissue 38 is realized by adjusting the MION-46 dosage and adjusting the echo time (TE) of ²³Na MRI.

Further, it is preferable that the MION-46 dose and TE are optimized based on signal-to-noise ratio (SNR) loss, susceptibility induced loss and blood signal attenuation. When the MION-46 dosage and the echo time are appropriately selected, there may be a reduction in blood pool signal of about 61%.

Next, ²³Na MRI is performed on the heart of one who has had MION-46 administered intravenously (step 430). The intravenously administered MION-46 reduces the blood signal intensity in sodium images (i.e., ²³Na MRI).

One possible explanation for the decrease in blood signal intensity may be due to the modification of the short and long transverse relaxation time components of sodium. The MION-46 dextran particles are T₂-relaxants causing decrease contrast between blood and ventricular muscle, by decreasing the ²³Na signal from blood plasma. As a result of the blood signal intensity suppression of the blood plasma signal, there is a reduction in ²³Na signal corresponding to the blood in the ventricular cavities 32, 34. Thus, ²³Na MRI after the intravenous administration of MION-46 provides decreased signal intensity differences between the ventricular cavities 32,34 and the ventricular wall 36 (FIG. 3) as compared with ²³Na MRI without administration of MION-46 where there is minimal signal difference between the ventricular cavities 12, 14 and the ventricular wall 16 (FIG. 1).

In addition to suppressing the blood signal associated with the ventricular cavities 32, 34, MION-46 also reduces the signal observed from the ventricular wall 36 due to blood perfusion of the ventricular wall 36 by the intravenously administered MION-46. The hypointense ²³Na signal for ventricular wall 36 and a more substantial hypointense signal of the blood signal in the ventricular cavities 32, 34, due to MION-46 administration, produces a ²³Na MRI image with a maximum visualization of the non-viable infarcted myocardial tissue 28 with a minimal signal associated with ventricular cavities 32, 34 and ventricular wall 36. Consequently, post-contrast imaging using optimal MION-46 doses and echo times (TE) produces distinct delineation of the myocardial infarction 38.

While the present invention was described showing the enhanced contrast between the ventricular cavities 22, 24, and infarcted myocardium 28, similar enhancements in contrast may be realized between atrial cavities and nonviable infarcted myocardium.

The use of a paramagnetic intravascular contrast agent such as MION-46 may have potential additional uses in addition to providing enhanced contrast in myocardial infarction detection in ²³Na MRI. A contrast agent may be used for assessment of myocardial blood perfusion volume at rest and after following a vasodilatory stimulus response.

A contrast agent may be administered to a subject may any of a number of routes. Intravenous administration is a preferred administration route. After the contrast agent has been administered, it is generally preferred to delay several minutes before imaging for the administered agent to reside in imaging effective amounts in cardiac tissue. Other administration routes also will be suitable, including direct injection into coronary tissue. A variety of subjects will be suitable for diagnosis and treatment in accordance with the invention, and typically are mammals, including primates, especially humans.

Optimal dosage amounts for any particular contrast agent and subject can be readily determined empirically. That is, the agent can be administered to the subject and the subject evaluated.

All documents mentioned herein are incorporated herein by reference. The following non-limiting example is illustrative of the invention.

### EXAMPLE

Experiments were performed and data were collected employing the present method in a canine model of myocardial infarction. Specifically, the experiments were directed to evaluating variation in the blood T₁ and T₂ relaxation times with increasing MION-46 amounts and determining the optimal dose for contrast-enhanced ²³Na cardiac MRI in normal canine hearts in vivo.

All MRI studies were performed with a commercially available 1.5T MRI system (Signa, Horizon, Echo Speed, 5.7 Epic platform, GE Medical Systems, Milwaukee, Wis.) equipped with broadband spectroscopy capabilities, using a 16-pole quadrature ²³Na birdcage coil tuned to 16.89 MHz and interfaced with a quadrature hybrid splitter. ²³Na MRI was performed with a three-dimensional (3D) twisted projection imaging (TPI) sequence using conventional scanner hardware with gradient having a maximum amplitude of 2.2 mT/m, a maximum slew rate of 12 mT/cm/s, and with a slew rate duty cycle limit of 20%. The k-space traversals traced the surfaces of concentric cones contained inside a sphere of radius kₘₐₓ. The desired gradient waveforms were computed from the k-space traversals and were used to encode the free induction decay.

In vitro studies were performed with a co-axial cylindrical plexiglass phantom of outer diameter 12 cm, and inner diameter 5 cm. A saline gel was prepared by mixing 4% per wt. of agarose into 500 ml of 65 mM saline in doubly de-ionized water, doped with 10 mM of copper sulphate (CuSO₄) and poured into the outer annular compartment of the phantom. This agarose gel provides a reasonable approximation to the ²³Na MRI relaxation properties of myocardial tissue. Whole blood samples were prepared by adding heparin (with 8.6 mg of sodium per ml of heparin solution) to canine blood with predetermined amounts of MION-46, and placed in the inner cylindrical chamber of the phantom.

Conditioned mongrel dogs weighing 25-30 kg were sedated with 10 mg/kg ketamine, 2.4 mg/kg xylanine, and 0.02 mg/kg atropine intramuscularly. An intravenous catheter was introduced, and the dog anesthetized with sodium penthothal, intubated, mechanically ventilated with 100% oxygen (tidal volume=10-15 ml/kg, respiratory rate=10-12 bpm) and maintained on general anesthesia with 1-2% isoflurane. Three dogs were studied as normal controls. Three dogs were subjected to a 90 to 120-minute balloon occlusion of the left anterior descending (LAD) coronaxy artery followed by reperfusion. A femoral artery cutdown was performed to place a 8 Fr. catheter introducer sheath (Terumo Medical Corp., Elkton, Md.) and a 7 Fr. right Judkins catheter (Cordis, Diamond Bar, Calif.) with a 0.014 mm guidewire and 3.5 Fr. coronary angioplasty balloon into the left anterior descending coronary artery under fluoroscopic guidance. The balloon was deflated and the occluder removed to allow reperfusion 3 to 6 hours prior to the MRI studies. MION-46 (42 mg Fe/ml, Advanced Magnetics Inc., Cambridge, Mass.) was injected intravenously in normal and in infarcted animals prior to contrast MRI. After MRI, infarcted dogs were humanely euthanized and the heart excised, sliced along the axial planes, and incubated in triphenyl-tetrazolium chloride (TTC) to identify viable from non-viable myocardium.

Dogs were positioned in the magnet in right decubitus, and an ECG-gated multislice gradient echo-transaxial ¹H MRI was performed with a flexible, 4-element phased array coil after autoshimming. The ¹H phased-array coil was then replaced by the ²³Na birdcage coil without repositioning the animal. The ²³Na three-dimensional twisted projection imaging (3D) (TPI) sequence was applied with TE=0.37 ms, TR=60 ms, 14 NEX, and a receiver bandwidth of 31.25 kHz, 1240 projections and gradient strengths up to 0.16 G/cm. To maximize detection of the short T₂ component, a 0.4 ms non-selective 90° radio frequency (rf) excitation pulse was used. Post-contrast ²³Na MRI was performed (TE=0.37 ms, 5 ms) at increasing MION-46 doses in normal dogs, and in infarcted animals at the target dose level of 10 mg/kg body weight (as determined from the normal dog experiments) to produce the greatest suppression of ventricular blood. The scan time per acquisition was approximately 17 minutes.

To determine the variation of the ²³Na relaxation times of blood with the amount of MION-46 injected, T₁ and T₂ measurements were carried out using saturation recovery (TR=60-300 ms) and spin-echo spectroscopic sequences (TE=12-35 ms) at increasing MION amounts ranging from 0-0.25 ml. The acquisition parameters were; 8-32 kHz, 256-512 points, NEX=96. T_{2f} and T₂ₛ and image contrast were also measured from sequentially acquired ²³Na TPI images with TE=0.37-17 ms, TR=55 ms, NEX=4, in 4 min. of scan time per acquisition.

Magnetic resonance spectroscopy (MRS) data from the phantom experiments were processed for peak area measurements. Selected peaks from blood spectra were fitted in the frequency domain by fitting Lorentzian or Gaussian functions and the areas computed by integration. Raw TPI data were reconstructed off-line on a Silicon Graphics (SGI) workstation using a regridding algorithm and 3D Fourier transformation.

To determine the T_{2f}, T₂ₛ and image signal intensity as a function of MION-46 amount, mean signal intensities were measured in multiple regions of interest (ROI) in sequentially acquired phantom and heart images (reconstructed at different TE values that ranged from 0.37-20 ms) with software developed previously. T_{2f} and T₂ values were determined by fitting image intensities (measured over 20-30 voxels within selected ROIs) and peak spectral areas as a function of TE to double exponential curves using a nonlinear least squares procedure based on the Marquardt-Levenberg algorithm (Multifit, Version 2.0, Day Computing, Cambridge, UK).

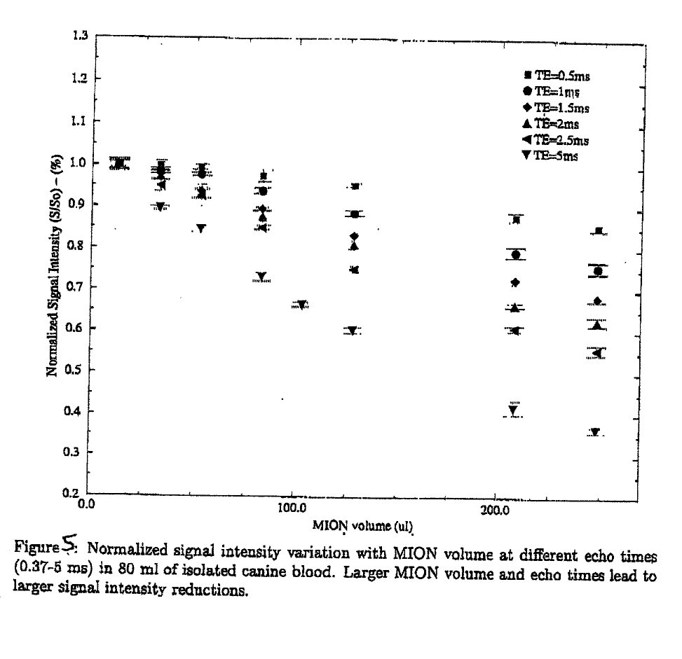

FIG. 5 depicts the ²³Na signal intensity normalized by the pre-contrast intensity of isolated canine blood (approximately 80 ml) as a function of MION-46 quantity (0-0.25 ml of 42 mg/ml of MION-46) and TE in the two compartment gel-blood cylindrical phantom. The MION-46 was mixed with blood to ensure a homogeneous distribution in the compartment. ²³Na MRI performed at TE values of 0.37-5 ms showed larger decreases in normalized blood signal intensity at long echo times and larger MION-46 volumes, reaching a 65% decrease at TE=5 ms for an administered MION-46 volume of 0.25 ml. Although the signal intensity decreased monotonically with increasing MION-46 volumes and longer TE values, signal-to-noise (SNR) and susceptibility induced losses also increased.

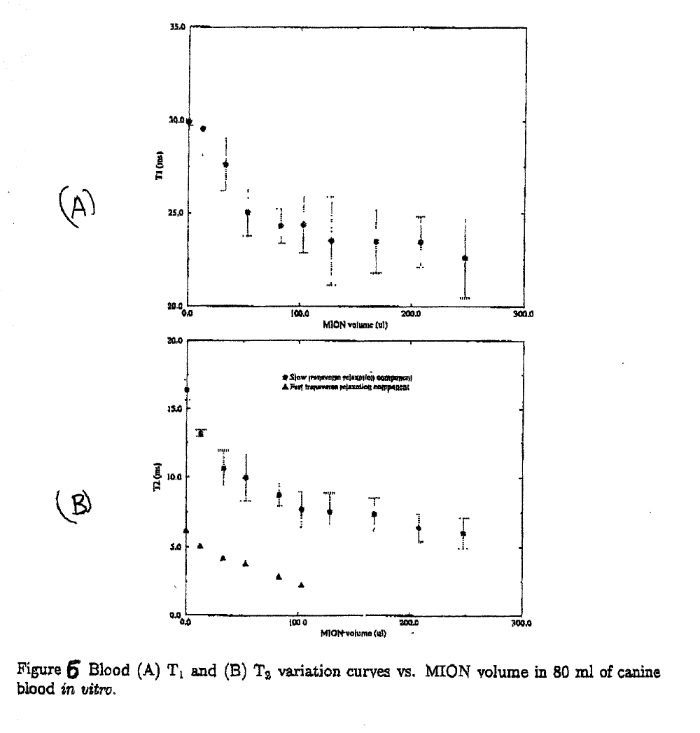

FIG. 6 shows the sodium T₁, T_{2f}, and T₂ₛ in the isolated blood phantom as a function of MION-46 volume in isolated, heparinized blood at 37° C. Maximum effect on both T₁ and T₂ is observed at larger values, although MION-46 volumes exceeding about 100 μl do not elicit further significant reductions in relaxation times. MION-46 administration in isolated blood caused a T₁ decrease from 30 ms (no MION-46) to 23 ms with 0.25 ml of MION-46. The fractional T₂ reductions were more prominent (FIG. 6B) With T_{2f} changing from 6.2 ms (no MION-46) to 2.3 ms with 0.1 ml of MION-46, and T₂ₛ from 16.4 ms to 6 ms with 0.25 ml of MION-46.

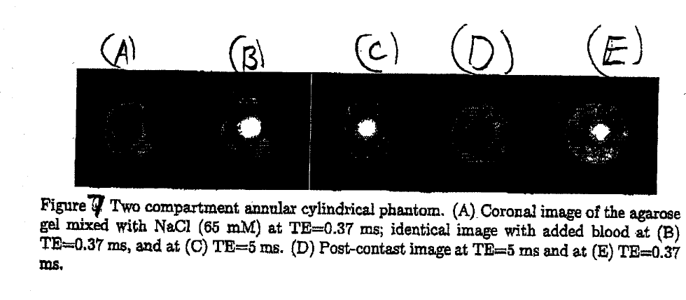

A series of coronal images from the gel-phantom is shown in FIG. 7. FIG. 7A shows an image of the gel phantom without blood at TE=0.37 ms, whereas FIG. 7B and FIG. 7C show images at TE=0.37 ms and at TE=5 ms, respectively, with blood added but no MION-46. FIG. 7D shows an image of the same phantom at TE=5 ms at a MION-46 volume of 0.25 ml. The blood signal intensity is almost completely eliminated. On the other hand, the same phantom with the same MION-46 value imaged at TE=0.37-ms reveals the blood-filled inner compartment (FIG. 7E). In general, SNR values in blood and gel regions of the phantom ranged from 10-24. Contrast-to-noise ratio (CNR) decreased from 9.3 (no MION-46) to −11.5 at the administered MION-46 volume of 0.25 ml. T₁ and T₂ measurements from gel regions of the phantom were found to be 30.3 ms and 38.4 ms, respectively.

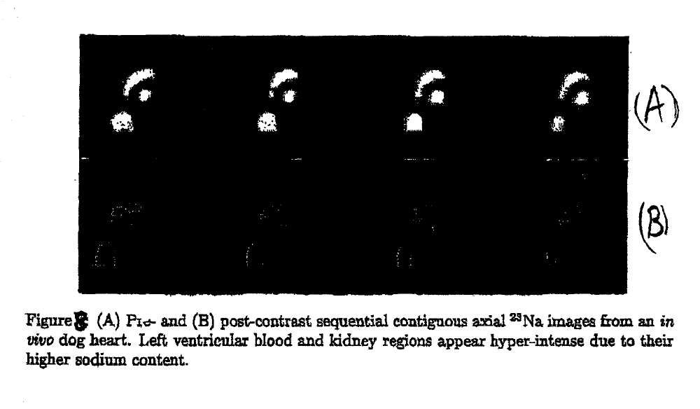

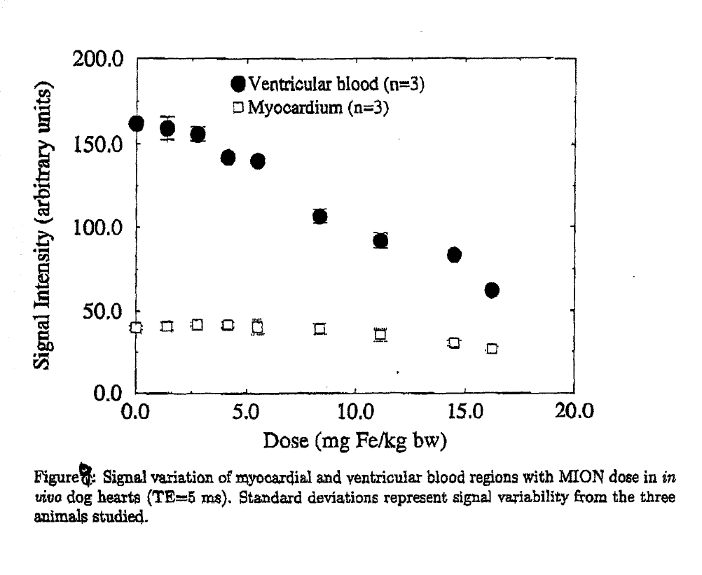

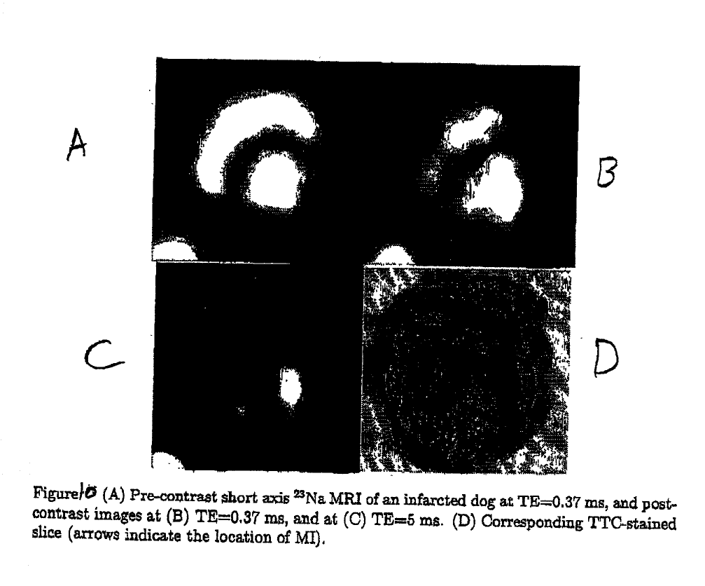

Typical pre- and post-contrast axial images from a normal dog heart are shown in FIG. 8. The contrast between the ventricular blood and normal (i.e. viable well-perfused) myocardium decreased with larger MION-46 doses as shown in FIG. 9. A dose of 10 mg/kg bw was chosen as optimal, since the image quality was compromised at higher doses by susceptibility-induced losses (FIG. 7B). Imaging at such a dose translated to a mean 61% decrease in blood signal decrease in the ventricular cavity in the normal dogs. Signal decrease was also evident in the liver. Pre- and post-contrast images at TE of 0.37 ms and 5 ms and dose values of 10 mg/kg of MION-46 in an infarcted dog heart are shown in FIG. 10. Administration of MION-46 resulted in signal dropout in all myocardial areas except the anterior wall (FIG. 10C) which corresponds to the infarcted or TTC-negative region (FIG. 10D). In this animal, the CNR between the infarct and the ventricular blood pool in sodium images was 11.2 pre-contrast, compared to −25.2 post-contrast, representing an 325% contrast improvement.

Based on the experiments performed and data collected, the following conclusions were made. Blood plasma is a major contributor to the sodium signal in cardiac ²³Na MRI. The administration of MION-46 to the blood pool reduces both the T₁ and T₂ relaxation times in ²³Na MRI. Based on the data presented here, the mechanism of action is believed to be due to susceptibility induced reductions of the fast and slow transverse relaxation components. The results from both in vitro and in vivo blood experiments demonstrate the importance of choice of both the echo time, and MION-46 concentration in optimizing the reduction in blood ²³Na signal decrease. The phantom experiments with isolated blood showed that the best strategy for maximum reduction in blood signal intensity is to adjust TE to eliminate the T₂ₛ signal component, as demonstrated in FIG. 6. Almost complete elimination of the blood signal is observed after administration of MION-46 volume of 0.25 ml in approximately 80 ml of blood in images acquired with TE=5 ms compared to those acquired at a TE of 0.37 ms, consistent with the fact that both the T_{2f} and T₂ₛ were reduced to 6.5 ms or less at this MION-46 volume.

The contrast between the myocardial wall and ventricular blood was decreased from about 4-fold to 2-fold with MION-46 administration at a dose of 10 mg Fe/kg body weight. Dose and TE choices were optimized based on SNR, susceptibility induced losses and blood signal attenuation. Since susceptibility induced artifacts are also tissue/organ geometry dependent, becoming worse at longer TE values, attention was paid to orient the short axis of the heart with the external magnetic field and to choose the minimum possible TE to attain maximum contrast. Note that the myocardial signal is reduced by approximately 25% at larger doses, possibly due to the expected 10-15% of blood volume signal in the myocardial capillary bed that may be completely or partially eliminated by the contrast agent. It remains to be determined whether the intravascular sodium affected by MION-46 administration, is due to fast exchange of sodium across blood vessel walls or extension of the large susceptibility gradients into the extravascular space.

Macromolecular MRI contrast agents may have toxic effects. No adverse toxic reactions have been observed in this study for the range of the injected doses administered (0-17 mg Fe/kg body weight); there were no changes in heart rate, blood pressure or anesthetic depth.

These experiments provide support for the use of the contrast agent MION-46 for enhancing the detection of myocardial infarction in vivo by ²³Na MRI and for the utility of the using MION-46 to diagnose heart disease.

These experiments provide support for the practical use of the contrast agent for clinical applications in ischemic heart disease. Although the agent caused large reductions in the signal intensity in the ventricular blood and smaller reductions in normal myocardium, it allowed the clear differentiation of non-viable infarcted tissue. Use of MION-46 may provide a useful method for enhancing the noninvasive identification of the location and extent of myocardial infarction by ²³Na MRI. Its applications and use might be extended to the study of myocardial perfusion at rest and after a vasodilation stimulus for the evaluation of isehemic disease. Furthermore, since the drug distribution depends on particle size, it will be important in future studies of myocardial infarction to relate the volume of distribution of the drug to the area of ²³Na image enhancement to assess the extent of the injury.

Although the invention has been described above in relation to preferred embodiments thereof, it will be understood by those skilled in the art that variations and modifications can be effected in these preferred embodiments without departing from the scope and spirit of the invention.

What is claimed is:

1. A method for evaluating biological tissue, comprising:
a) treating biological tissue with an iron oxide contrast agent; and
b) imaging the tissue with ²³Na or ³⁹K magnetic resonance.

2. The method of claim 1 wherein the tissue is imaged with ²³Na MRI.

3. The method of claim 1 wherein the tissue is imaged with ³⁹K MRI

4. The method of any one of claims 1 through 3 wherein the tissue is cardiac tissue.

5. The method of claim 1 through 3 wherein the tissue comprises infarcted cardiac tissue.

6. The method of any one of claim 1 through 5 further comprising assessing the MRI image to detect infracted tissue.

7. The method of any one of claims 1 through 6 wherein the contrast agent comprises one or more iron atoms coordinated with a polymer.

8. The method of any claims 1 through 6 wherein the contrast agent comprises one or more iron atoms coordinated with a polymer having oxygen substitution.

9. The method of any one of claims 1 through 6 wherein the contrast agent comprises one or more iron atoms coordinated with a polysaccharide.

10. The method of any one of claims 1 through 6 wherein the contrast agent comprises one or more iron atoms coordinated with a dextran.

11. The method of any one of claims 1 through 6 wherein the contrast agent is MION-46.

12. The method of any one of claims 1 through 11 wherein the contrast agent is administered to a subject suffering from or susceptible to myocardial infarction.

13. The method of claim 12 further comprising selecting a subject suffering or susceptible to myocardial infarction and then administering the contrast agent to the selected subject.

14. The method of any one of claims 1 through 13 wherein the contrast agent is administered to a subject and a magnetic resonance study is made of the subject's heart.

15. The method of claim 14 wherein the magnetic resonance study differentiates between normal myocardial tissue, injured myocardial tissue and infarcted myocardial tissue.

16. A method identifying infarcted myocardial tissue of a subject comprising:
a) administering to the subject an imaging-effective amount of an iron oxide contrast agent; and
b) imaging the subject's heart with ²³Na or ³⁹K magnetic resonance.

17. The method of claim 16 wherein the subject is suffering from or has suffered cardiac disorder.

18. The method of claim 16 or 17 wherein the subject is suffering from or has suffered heart failure of cardiogenic shock.

19. The method of claim 16 or 17 wherein the subject is suffering from or has suffered a cardiovascular disorder.

20. The method of any one of claims 16 through 19 wherein the tissue is imaged with ²³Na MRI.

21. The method of any one of claims 16 through 19 wherein the tissue is imaged with ³⁹K MRI.

22. The method of any one of claims 16 through 21 wherein the contrast agent comprises one or more iron atoms coordinated with a polymer.

23. The method of any claims 16 through 21 wherein the contrast agent comprises one or more iron atoms coordinated with a polymer having oxygen substitution.

24. The method of any one of claims 16 through 21 wherein the contrast agent comprises one or more iron atoms coordinated with a polysaccharide.

25. The method of any one of claims 16 through 21 wherein the contrast agent comprises one or more iron atoms coordinated with a dextran.

27. The method of any one of claims 16 through 21 wherein the contrast agent is MION-46.

28. A magnetic resonance system comprising:
a magnetic resonance imaging apparatus for ²³Na or ³⁹K imaging; and an iron oxide contrast agent.

29. The system of claim 28 wherein the system is adapted for ²³Na imaging.

30. The system of claim 28 wherein the system is adapted for ³⁹K imaging.

31. The system of any one of claims 28 through 30 wherein the contrast agent comprises one or more iron atoms coordinated with a polymer.

32. The system of any one of claims 28 through 30 wherein the contrast agent comprises one or more iron atoms coordinated with a polymer having oxygen substitution.

33. The system of any one of claims 28 through 30 wherein the contrast agent comprises one or more iron atoms coordinated with a polysaccharide.

34. The system of any one of claims 28 through 30 wherein the contrast agent comprises one or more iron atoms coordinated with a dextran.

35. The system of any one of claims 28 through 30 wherein the contrast agent is MION-46.

36. The system of any one of claims 28 through 35 wherein the contrast agent is packaged in a pharmaceutically acceptable form.
